SEQUENCE LISTING (20 sequences)

SEQ ID NO: 1 - DNA, 29 bp, Artificial Sequence
  Synthetic Oligonucleotide
cagctgcacgctgccgtctaaaaaaaaaa

SEQ ID NO: 2 - DNA, 28 bp, Artificial Sequence
  Synthetic Oligonucleotide
cagctgcacgctgccgtcaaaaaaaaaa

SEQ ID NO: 3 - DNA, 24 bp, Artificial Sequence
  Synthetic Oligonucleotide
cagctgcacgctgcaaaaaaaaaa

SEQ ID NO: 4 - DNA, 24 bp, Artificial Sequence
  Synthetic Oligonucleotide
cagctgcacgctgcaaaaaaaaaa

SEQ ID NO: 5 - DNA, 28 bp, Artificial Sequence
  Synthetic Oligonucleotide.
cagctgcacgctgccgtcaaaaaaaaaa

SEQ ID NO: 6 - DNA, 24 bp, Artificial Sequence
  Synthetic Oligonucleotide
tttttcagctgcacaaaaaaaaaa

SEQ ID NO: 7 - DNA, 29 bp, Artificial Sequence
  Synthetic Oligonucleotide.
tttttcagctgcacgctgcaaaaaaaaaa

SEQ ID NO: 8 - DNA, 29 bp, Artificial Sequence
  Synthetic Oligonucleotide
ttttttttttcagctgcacaaaaaaaaaa

SEQ ID NO: 9 - DNA, 29 bp, Artificial Sequence
  Synthetic Oligonucleotide
tttttttttttttttttttaaaaaaaaaa

SEQ ID NO: 10 - DNA, 30 bp, Artificial Sequence
  Synthetic Oligonucleotide
tttttttttttttttttttttttttttttt

SEQ ID NO: 11 - DNA, 29 bp, Artificial Sequence
  Synthetic Oligonucleotide
ccccccagctgcacgctgcaaaaaaaaaa

SEQ ID NO: 12 - DNA, 29 bp, Artificial Sequence
  Synthetic Oligonucleotide
cccccccccccagctgcacaaaaaaaaaa

SEQ ID NO: 13 - DNA, 29 bp, Artificial Sequence
  Synthetic Oligonucleotide
cccccccccccccccccccaaaaaaaaaa

SEQ ID NO: 14 - DNA, 30 bp, Artificial Sequence
  Synthetic Oligonucleotide
cccccccccccccccccccccccccccccc

SEQ ID NO: 15 - DNA, 29 bp, Artificial Sequence
  Synthetic Oligonucleotide
aaaaacagctgcacgctgcaaaaaaaaaa

SEQ ID NO: 16 - DNA, 29 bp, Artificial Sequence
  Synthetic Oligonucleotide
aaaaaaaaaacagctgcacaaaaaaaaaa

SEQ ID NO: 17 - DNA, 29 bp, Artificial Sequence
  Synthetic Oligonucleotide
aaaaaaaaaaaaaaaaaaaaaaaaaaaaa

SEQ ID NO: 18 - DNA, 31 bp, Artificial Sequence
  Synthetic Oligonucleotide
aaaaaaaaaaaaaaaaaaaaaaaaaaaaaan

SEQ ID NO: 19 - DNA, 24 bp, Artificial Sequence
  Synthetic Oligonucleotide
cagctgcacgctgcaaaaaaaaaa

SEQ ID NO: 20 - DNA, 10 bp, Artificial Sequence
  Synthetic Oligonucleotide
aaaaaaaaaa

(19) United States
(12) Patent Application Publication
(10) Pub. No.: US 2017/0044544 A1
(43) Pub. Date: Feb. 16, 2017

(54) OLIGONUCLEOTIDE SPECIFIC UPTAKE OF NANOCONJUGATES

(71) Applicant: NORTHWESTERN UNIVERSITY, Evanston, IL (US)

(72) Inventors: Chad A. Mirkin, Wilmette, IL (US); David A. Giljohann, Chicago, IL (US)

(21) Appl. No.: 15/337,674

(22) Filed: Oct. 28, 2016

Related U.S. Application Data
(63) Continuation of application No. 13/518,443, filed on Oct. 3, 2012, now abandoned.
(60) Provisional application No. 61/290,123, filed on Dec. 24, 2009.

(51) Int. Cl. C12N 15/113

(52) U.S. Cl. CPC C12N 15/113; C12N 2310/11; C12N 2310/351

(57) ABSTRACT

Disclosed are nanoparticles functionalized with an oligonucleotide and a domain, wherein the domain increases cellular uptake of the nanoparticles. The domain is a sequence of nucleobases or phosphate groups, such as a poly thymidine (polyT) sequence or a phosphate polymer (C3 residue) and may be located 5′ to the oligonucleotide 3′ to the oligonucleotide, within, or colinear with the oligonucleotide. Usage of the nanoparticles including modulating gene regulation is contemplated.

### CROSS REFERENCE TO RELATED APPLICATIONS

This application claims the priority benefit under 35 U.S.C. §119(e) of U.S. Provisional Application No. 61/290,123, filed Dec. 24, 2009, the disclosure of which is incorporated herein by reference in its entirety.

### STATEMENT OF GOVERNMENT INTEREST

This invention was made with government support under Grant Number 5U54 CA 119341 awarded by the National Institutes of Health (NCI/CCNE) and Grant Number 5DPI OD000285 awarded by the National Institutes of Health (NIH). The government has certain rights in the invention.

### FIELD OF THE INVENTION

The present invention is directed to nanoparticles functionalized with an oligonucleotide and a domain that can affect the uptake of the nanoparticle by a cell.

### BACKGROUND OF THE INVENTION

Oligonucleotides are widely considered poor candidates for crossing cellular membranes due to the high negative charge resulting from their phosphate backbone. Thus, strategies for affecting cellular entry commonly include complexation with cationic transfection reagents. DNA-functionalized gold nanoconjugates (DNA-Au NPs) are a unique class of material consisting of a gold nanoparticle core that is functionalized with a dense shell of synthetic oligonucleotides. They are readily able to transverse cellular membranes, not requiring the addition of toxic transfection reagents. Importantly, these structures do not serve solely as vehicles for nucleic acid delivery, but exhibit cooperative properties that result from their polyvalent surfaces. Recent efforts to silence genes using DNA-Au NPs as well as siRNA-Au NPs have proven these conjugates to be highly promising agents for gene regulation and live cell mRNA detection. These structures exhibit enhanced cellular/tissue uptake with improved biostability and compatibility compared to conventional molecular DNA and RNA transfection methodologies.

### SUMMARY OF THE INVENTION

Described herein is a nanoparticle composition that comprises a domain that is useful for regulating the uptake of the nanoparticle into a cell. The composition described herein enters cells without transfection agents and the domain allows for control of the amount of nanoparticles that enters and remains in a cell.

Thus, in some embodiments a nanoparticle functionalized with an oligonucleotide and a domain is provided, the nanoparticle having the property of being taken up by a cell at an efficiency different than a nanoparticle functionalized with the same oligonucleotide but lacking the domain. In some aspects, the domain is located 5′ to the oligonucleotide. In some aspects, the domain is located 3′ to the oligonucleotide. In some aspects, the domain is located at an internal region within the oligonucleotide. In further aspects, the domain is colinear with the oligonucleotide.

In some embodiments, a nanoparticle is provided that is functionalized with a second oligonucleotide and a domain is associated with the second oligonucleotide.

In some embodiments, a nanoparticle is provided that comprises a domain wherein the domain comprises a polythymidine (polyT) sequence comprising more than one thymidine residue.

In further embodiments, a nanoparticle is provided that comprises a domain wherein the domain comprises a polythymidine (polyT) sequence comprising two thymidine residues.

In still further embodiments, a nanoparticle is provided that comprises a domain wherein the domain comprises a polythymidine (polyT) sequence comprising two, three, four, five, six, seven, eight, nine, ten, eleven, twelve, thirteen, fourteen, fifteen, sixteen, seventeen, eighteen, nineteen, or twenty thymidine residues.

In some aspects a nanoparticle is provided comprising a domain wherein the domain comprises a phosphate polymer (C3 residue). In some aspects, the domain comprises two or more phosphate polymers (C3 residues).

In some embodiments a method of modulating cellular uptake capacity of an oligonucleotide-functionalized nanoparticle is provided comprising the step of modifying the nanoparticle to further comprise a domain that modulates cellular uptake of the oligonucleotide-functionalized nanoparticle compared to the oligonucleotide-functionalized nanoparticle lacking the domain. In some aspects the domain increases cellular uptake of the functionalized nanoparticle. In some aspects the domain comprises a polyT sequence comprising more than one thymidine residue. In further aspects, the domain comprises a polyT sequence comprising two, three, four, five, six, seven, eight, nine, ten, eleven, twelve, thirteen, fourteen, fifteen, sixteen, seventeen, eighteen, nineteen, or twenty thymidine residues.

Methods are herein provided wherein an increase in thymidine residues in the polyT sequence of the first oligonucleotide-functionalized nanoparticle increases cellular uptake compared to the second oligonucleotide-functionalized nanoparticle that does not contain the polyT sequence.

In some embodiments, the domain decreases cellular uptake of the oligonucleotide-functionalized nanoparticle. In some aspects, the domain comprises a phosphate polymer (C3 residue).

Methods are thus provided wherein an increase in C3 residues on the first oligonucleotide-functionalized nanoparticle decreases cellular uptake compared to the second oligonucleotide-functionalized nanoparticle that does not contain a C3 residue.

Any of the methods disclosed herein are contemplated for use with a nanoparticle likewise described herein.

### BRIEF DESCRIPTION OF THE DRAWINGS

FIG. 1 depicts the synthesis and characterization of nanoconjugates.

FIG. 2 depicts cellular uptake (particles/cell) for nanoconjugates lacking nucleobases.

FIG. 3 depicts cellular uptake as a function of location of phosphate (C3) backbone.

FIG. 4 depicts a comparison of cellular uptake for conjugates containing poly thymidine repeats.

### DETAILED DESCRIPTION OF THE INVENTION

A property of DNA-Au NPs is their ability to enter a wide variety of cell types as a result of the dense functionalization of oligonucleotides on the nanoparticle surface. These cell types include eukaryotic and prokaryotic cells. Those of skill in the art will understand that all eukaryotic and prokaryotic cell types are contemplated for use in the methods disclosed herein. The facile uptake of these structures into cells was not predicted, given that these structures contain a densely functionalized shell of polyanionic oligonucleotides, and that strategies for the introduction of oligonucleotides into cells typically requires that the oligonucleotide is complexed with positively charged agents in order to effect cellular internalization. It has been shown in all cell types examined to date (see Table 1, below), including primary cells and tissues, that both DNA-Au NPs and RNA-Au NPs can be added directly to cell culture media and are subsequently taken up by cells in high numbers.

TABLE 1

| Cell Type | Designation or Source |
|---|---|
| Breast | SKBR3, MDA-MB-321, AU-565 |
| Brain | U87, LN229 |
| Bladder | HT-1376, 5637, T24 |
| Colon | LS513 |
| Cervix | HeLa, SiHa |
| Skin | C166, KB, MCF, 10A |
| Kidney | MDCK |
| Blood | Sup T1, Jurkat |
| Leukemia | K562 |
| Liver | HepG2 |
| Kidney | 293T |
| Ovary | CHO |
| Macrophage | RAW 264.7 |
| Hippocampus Neurons | primary, rat |
| Astrocytes | primary, rat |
| Glial Cells | primary, rat |
| Bladder | primary, human |
| Erythrocytes | primary, mouse |
| Peripheral Blood | primary, mouse |
| Mononuclear Cell | |
| T-Cells | primary, human |
| Beta Islets | primary, mouse |
| Skin | primary, mouse |

It has previously been determined that oligonucleotide density on the surface plays a key role in mediating cellular uptake, however, it is unclear how oligonucleotides are involved in this process.

Another contribution of oligonucleotides to the uptake of nanoparticle conjugates is disclosed herein. Specifically, nucleobases of the oligonucleotide are demonstrated to be the contributing factor to cellular uptake. In addition, specific domains are identified that either enhance or reduce cellular uptake. As is understood in the art, polyvalent oligonucleotide-Au NPs have unique size, charge, and surface functionality, with properties derived from the combination of the oligonucleotides and the Au NP. To test the contribution of the oligonucleotides present on the nanoparticle surface to their cellular uptake, the entry of particles was examined as a function of nucleobase structure and sequence.

The present disclosure demonstrates the utility of an oligonucleotide-functionalized nanoparticle, wherein the oligonucleotide further comprises a domain which modulates cellular uptake. As used herein, a “domain” is understood to be a sequence of nucleobases or phosphate groups. Modified nucleobases as defined herein are also contemplated to make up a domain as provided herein. A domain is in one aspect collinear with an oligonucleotide functionalized on a nanoparticle. In another aspect, the domain is associated directly with the nanoparticle, absent association with an oligonucleotide functionalized on the nanoparticle. In still another aspect, the domain is associated with the nanoparticle through a spacer, and absent association with an oligonucleotide functionalized on the nanoparticle (i.e., the domain is in some aspects associated with the nanoparticle through a spacer, separate from any association with an oligonucleotide).

It is noted here that, as used in this specification and the appended claims, the singular forms “a,” “an,” and “the” include plural reference unless the context clearly dictates otherwise.

It is to be noted that the terms “polynucleotide” and “oligonucleotide” are used interchangeably herein and have meanings accepted in the art.

It is further noted that the terms “attached”, “conjugated” and “functionalized” are also used interchangeably herein and refer to the association of an oligonucleotide or domain with a nanoparticle.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

### Nanoparticles

Nanoparticles are provided which are functionalized to have a polynucleotide attached thereto. The size, shape and chemical composition of the nanoparticles contribute to the properties of the resulting polynucleotide-functionalized nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37(1999); Marinakos et al., Chem. Mater. 10: 1214-19(1998); Enustun & Turkevich, J. Am. Chem. Soc. 85: 3317(1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

### Oligonucleotides

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nuclcotidcs that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. Sec, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof, and a domain as defined herein, generally comprise a polynucleotide from about 5 nucleotides to about 100 nucleotides in length. More specifically, nanoparticles are functionalized with polynucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the oligonucleotide attached to a nanoparticle is DNA. When DNA is attached to the nanoparticle, the DNA is comprised of a sequence that is sufficiently complementary to a target region of a polynucleotide such that hybridization of the DNA oligonucleotide attached to a nanoparticle and the target polynucleotide takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the oligonucleotide functionalized on the nanoparticle can form a triplex structure with a double-stranded target polynuclcotide. In another aspect, a triplex structure can be formed by hybridization of a double-stranded oligonucleotide functionalized on a nanoparticle to a single-stranded target polynucleotide.

In some embodiments, the disclosure contemplates that a polynucleotide attached to a nanoparticle is RNA. In some aspects, the RNA is a small interfering RNA (siRNA).

Oligonucleotides, as defined herein, also includes aptamers. In general, aptamers arc nucleic acid or peptide binding species capable of tightly binding to and discreetly distinguishing target ligands [Yan et al., RNA Biol. 6(3) 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990). U.S. Pat. No. 5,270,163, and U.S. Pat. No. 5,637,459, each of which is incorporated herein by reference in their entirety]. General discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). Additional discussion of aptamers, including but not limited to selection of RNA aptamers, selection of DNA aptamers, selection of aptamers capable of covalently linking to a target protein, use of modified aptamer libraries, and the use of aptamers as a diagnostic agent and a therapeutic agent is provided in Kopylov et al., Molecular Biology 34(6): 940-954 (2000) translated from Molekulyamaya Biologiya, Vol. 34, No. 6, 2000, pp. 1097-1113, which is incorporated herein by reference in its entirety. In various aspects, an aptamer is about 10 to about 100 nucleotides in length, or about 100 to about 500 nucleotides in length.

The production and use of aptamers is known to those of ordinary skill in the art.

In some aspects, multiple oligonucleotides are functionalized to a nanoparticle. In various aspects, the multiple oligonucleotides each have the same sequence, while in other aspects one or more oligonucleotides have a different sequence. In further aspects, multiple oligonucleotides are arranged in tandem and are separated by a spacer. Spacers are described in more detail herein below.

Polynucleotides contemplated for attachment to a nanoparticle include those which modulate expression of a gene product expressed from a target polynucleotide. Polynucleotides contemplated by the present disclosure include DNA, RNA and modified forms thereof as defined herein below. Accordingly, in various aspects and without limitation, polynucleotides which hybridize to a target polynucleotide and initiate a decrease in transcription or translation of the target polynucleotide, triple helix forming polynucleotides which hybridize to double-stranded polynucleotides and inhibit transcription, and ribozymes which hybridize to a target polynucleotide and inhibit translation, are contemplated.

In various aspects, if a specific polynucleotide is targeted, a single functionalized oligonucleotide-nanoparticle composition has the ability to bind to multiple copies of the same transcript. In one aspect, a nanoparticle is provided that is functionalized with identical polynucleotides, i.e., each polynucleotide has the same length and the same sequence. In other aspects, the nanoparticle is functionalized with two or more polynucleotides which are not identical, i.e., at least one of the attached polynucleotides differ from at least one other attached polynucleotide in that it has a different length and/or a different sequence. In aspects wherein different polynucleotides are attached to the nanoparticle, these different polynucleotides bind to the same single target polynucleotide but at different locations, or bind to different target polynucleotides which encode different gene products.

### Domain

The domain that is part of the oligonucleotide-functionalized nanoparticle as described herein is shown to affect the efficiency with which the nanoparticle is taken up by a cell. Accordingly, the domain increases or decreases the efficiency. As used herein, “efficiency” refers to the number, amount or rate of uptake of nanoparticles in/by a cell. Because the process of nanoparticles entering and exiting a cell is a dynamic one, efficiency can be increased by taking up more nanoparticles or by retaining those nanoparticles that enter the cell for a longer period of time. Similarly, efficiency can be decreased by taking up fewer nanoparticles or by retaining those nanoparticles that enter the cell for a shorter period of time.

The domain, in some aspects, is located 5′ to the oligonucleotide. In some aspects, the domain is contiguous/colinear with the oligonucleotide and is located 3′ to the oligonucleotide. In further aspects, the domain is colinear with the oligonucleotide. In some aspects, the domain is located at an internal region within the oligonucleotide. In further aspects, the domain is located on a second oligonucleotide that is attached to a nanoparticle. In one aspect, more than one domain is present in an oligonucleotide functionalized to a nanoparticle. Accordingly, in some aspects more than one domain is present in tandem at the 5′ end, and/or at the 3′ end, and/or at an internal region of the oligonucleotide.

In another aspect, a domain, in some embodiments, is contemplated to be attached to a nanoparticle as a separate entity from an oligonucleotide, i.e., in some embodiments the domain is directly attached to the nanoparticle, separate from an oligonucleotide.

It is further contemplated that an oligonucleotide, in some embodiments, comprise more than one domain, located at one or more of the locations described herein.

The domain, in some embodiments, increases the efficiency of uptake of the oligonucleotide-functionalized nanoparticle by a cell. In some aspects, the domain comprises a sequence of thymidine residues (polyT) or uridine residues (polyU). In further aspects, the polyT or polyU sequence comprises two thymidines or uridines. In various aspects, the polyT or polyU sequence comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more thymidine or uridine residues.

In some embodiments, it is contemplated that a nanoparticle functionalized with an oligonucleotide and a domain is taken up by a cell with greater efficiency than a nanoparticle functionalized with the same oligonucleotide but lacking the domain. In some aspects, a nanoparticle functionalized with an oligonucleotide and a domain is taken up by a cell 1% more efficiently than a nanoparticle functionalized with the same oligonucleotide but lacking the domain. In various aspects, a nanoparticle functionalized with an oligonucleotide and a domain is taken up by a cell 2%, 3%, 4%, 5%, 6%, 7%, 8%, 9%, 10%, 11%, 12%, 13%, 14%, 15%, 16%, 178% 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold, about 150-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, about 2000-fold, about 2500-fold, about 3000-fold, about 3500-fold, about 4000-fold, about 4500-fold, about 5000-fold, about 5500-fold, about 6000-fold, about 6500-fold, about 7000-fold, about 7500-fold, about 8000-fold, about 8500-fold, about 9000-fold, about 9500-fold, about 10000-fold or higher, more efficiently than a nanoparticle functionalized with the same oligonucleotide but lacking the domain.

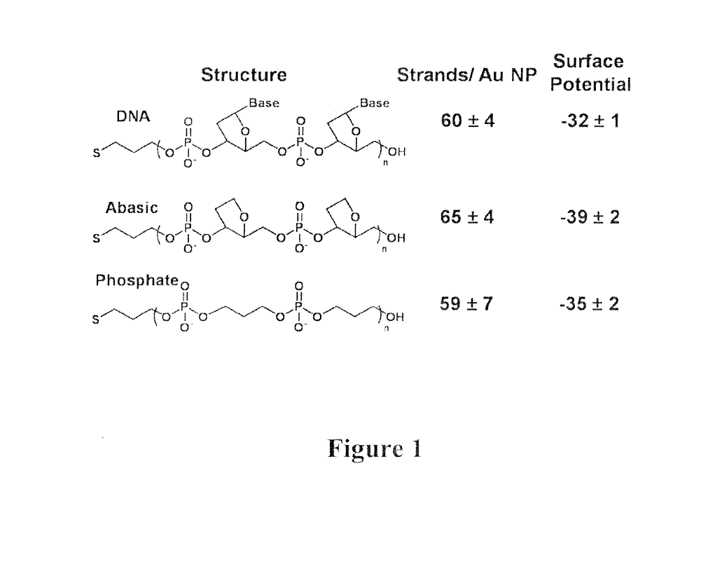

In some embodiments, the domain decreases the efficiency of uptake of the oligonucleotide-functionalized nanoparticle by a cell. In some aspects, the domain comprises a phosphate polymer (C3 residue; see FIG. 1) that is comprised of one phosphate. In various aspects, the C3 residue comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more phosphates.

In some embodiments, it is contemplated that a nanoparticle functionalized with an oligonucleotide and a domain is taken up by a cell with lower efficiency than a nanoparticle functionalized with the same oligonucleotide but lacking the domain. In some aspects, a nanoparticle functionalized with an oligonucleotide and a domain is taken up by a cell 1% less efficiently than a nanoparticle functionalized with the same oligonucleotide but lacking the domain. In various aspects, a nanoparticle functionalized with an oligonucleotide and a domain is taken up by a cell 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold, about 150-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, about 2000-fold, about 2500-fold, about 3000-fold, about 3500-fold, about 4000-fold, about 4500-fold, about 5000-fold, about 5500-fold, about 6000-fold, about 6500-fold, about 7000-fold, about 7500-fold, about 8000-fold, about 8500-fold, about 9000-fold, about 9500-fold, about 10000-fold or higher, less efficiently than a nanoparticle functionalized with the same oligonucleotide but lacking the domain.

### Modified Oligonucleotides

As discussed above, modified oligonucleotides are contemplated for functionalizing nanoparticles. In various aspects, an oligonucleotide functionalized on a nanoparticle is completely modified or partially modified. Thus, in various aspects, one or more, or all, sugar and/or one or more or all internucleotide linkages of the nucleotide units in the polynucleotide are replaced with “non-naturally occurring” groups.

In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. No. 4,981,957: 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂—, —O—, —S—, —NRH—, >C═O, >C═NRH, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH—CO—CH₂—, —CH—CHOH—CH₂—, —OCH₂—O—, —O—CH₂—CH₂—, —O—CH₂—CH=(including R5 when used as a linkage to a succeeding monomer), —CH₂—CH₂—, —NRH—CH₂—CH₂—, CH₂—CH—NRH—, —CH₂—NRH—CH₂—, —O—CH₂—CH₂—NRH—, —NRH—CO—, —NRH—CO—NRH—, —NRH—CS—NRH—, —NRH—C(═NRH)—NRH—, —NRH—CO—CH₂—NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O—CH₂—CH₂—NRH—, —CH═N—O—, —CH₂—NRH—O—, —CH₂—O—N=(including R5 when used as a linkage to a succeeding monomer), —CH₂—O—NRH—, —CO—NRH—CH₂—, —CH₂—NRH—O—, —CH₂—NRH—CO—, —O—NRH— CH₂—, —O—NRH, —O—CH₂—S—, —S— CH₂—O—, —CH₂—CH₂—S—, —O—CH₂— CH₂—S—, —S— CH₂—CH=(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂— CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O, —O—PO(NHRN)—O—, —O—P(O)₂—NRHH—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—P(—O,S)—O—, —O—P(S)₂—O—, —NRHP(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH2)ₙOCH₃, O(CH₂)ₙNH₂, O(CH₂)ₙCH₃, O(CH₂)ₙONH₂, and O(CH₂)ₙON[(CH₂)ₙCH₃], where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin el al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)n group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Oligonucleotide Attachment to a Nanoparticle

Oligonucleotides contemplated for use in the methods include those bound to the nanoparticle through any means. Regardless of the means by which the oligonucleotide is attached to the nanoparticle, attachment in various aspects is effected through a 5′ linkage, a 3′ linkage, some type of internal linkage, or any combination of these attachments.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety.

Nanoparticles with oligonucleotides attached thereto are thus provided wherein an oligonucleotide further comprising a domain is associated with the nanoparticle. In some aspects, the domain is a polythymidine sequence. In other aspects, the domain is a phosphate polymer (C3 residue).

### Spacers

In certain aspects, functionalized nanoparticles are contemplated which include those wherein an oligonucleotide and a domain are attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In aspects of the invention where a domain is attached directly to a nanoparticle, the domain is optionally functionalized to the nanoparticle through a spacer. In another aspect, the domain is on the end of the oligonucleotide that is opposite to the spacer. The arrangements of one or more of domain, spacer and oligonucleotide with respect to the nanoparticle to which each component (i.e., domain, spacer and oligonucleotide) is functionalized, either directly or indirectly, can be determined by one of ordinary skill in the art. In aspects wherein domains in tandem are functionalized to a nanoparticle, spacers are optionally between some or all of the domain units in the tandem structure. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the polynucleotides to become bound to the nanoparticles or to the target polynucleotide. The spacers should not have sequences complementary to each other or to that of the oligonucleotides, but may be all or in part complementary to the target polynucleotide. In certain aspects, the bases of the polynucleotide spacer are all adenines, all thymines, all cytidines, all guanines, all uracils, or all some other modified base. Accordingly, in some aspects wherein the spacer consists of all thymines or all uracils, it is contemplated that the spacer can function as a domain as described herein.

In some embodiments, spacer sequences of varying length are utilized to vary the number of and the distance between the RNA polynucleotides on a nanoparticle thus controlling the rates of target polynucleotide degradation. Without being bound by theory, one can control the rate of target polynucleotide degradation by immobilizing a RNA polynucleotide on a nanoparticle such that the protein interaction site is in a proximal position as described above. This aspect, combined with a surface density aspect as described below, can allow or prevent access by a polypeptide of the disclosure to the protein interaction site.

### Surface Density

Nanoparticles as provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell. The disclosure provides embodiments wherein the increased uptake of a nanoparticle due to a higher density of polynucleotides on the nanoparticle surface works in combination with the presence of a domain as described herein. For example and without limitation, a nanoparticle with a given density of polynucleotides on the surface of the nanoparticle, wherein the nanoparticle further comprises a polyT domain, will demonstrate an increased uptake of the functionalized nanoparticle by a cell relative to a nanoparticle with an identical density of polynucleotides on the surface of the nanoparticle, wherein the nanoparticle lacks a polyT domain. In various aspects, the increase in uptake by a cell of the functionalized nanoparticle further comprising the polyT domain is 1% relative to the functionalized nanoparticle lacking the polyT domain. In further aspects, the increase in uptake by a cell of the functionalized nanoparticle further comprising the polyT domain is 2%, 3%, 4%, 5%, 6%, 7%, 8%, 9%, 10%, 11%, 12%, 13%, 14%, 15%, 16%, 17%, 18%, 19%, 20%, 21%, 22%, 23%. 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold, about 150-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, about 2000-fold, about 2500-fold, about 3000-fold, about 3500-fold, about 4000-fold, about 4500-fold, about 5000-fold, about 5500-fold, about 6000-fold, about 6500-fold, about 7000-fold, about 7500-fold, about 8000-fold, about 8500-fold, about 9000-fold, about 9500-fold, about 10000-fold or higher relative to the functionalized nanoparticle lacking the polyT domain.

Likewise, a nanoparticle with a given density of polynucleotides on the surface of the nanoparticle, wherein the nanoparticle further comprises a C3 domain, will in various aspects demonstrate decreased uptake of the functionalized nanoparticle by a cell relative to a nanoparticle with an identical density of polynucleotides on the surface of the nanoparticle, wherein the nanoparticle lacks a C3 domain. In various aspects, the decrease in uptake by a cell of the functionalized nanoparticle further comprising the C3 domain is 1% relative to the functionalized nanoparticle lacking the C3 domain. In further aspects, the decrease in uptake by a cell of the functionalized nanoparticle further comprising the C3 domain is 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold, about 150-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, about 2000-fold, about 2500-fold, about 3000-fold, about 3500-fold, about 4000-fold, about 4500-fold, about 5000-fold, about 5500-fold, about 6000-fold, about 6500-fold, about 7000-fold, about 7500-fold, about 8000-fold, about 8500-fold, about 9000-fold, about 9500-fold, about 10000-fold or higher relative to the functionalized nanoparticle lacking the C3 domain.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

### Oligonucleotide Target Sequences and Hybridization

In some aspects, the disclosure provides methods of targeting specific nucleic acids. Any type of nucleic acid may be targeted, and the methods may be used, e.g., for therapeutic modulation of gene expression (See, e.g., PCT/US2006/022325, the disclosure of which is incorporated herein by reference). Examples of nucleic acids that can be targeted by the methods of the invention include but are not limited to genes (e.g., a gene associated with a particular disease), bacterial RNA or DNA, viral RNA, or mRNA, RNA, or single-stranded nucleic acids.

The terms “start codon region” and “translation initiation codon region” refer to a portion of a mRNA or gene that encompasses contiguous nucleotides in either direction (i.e., 5′ or 3′) from a translation initiation codon. Similarly, the terms “stop codon region” and “translation termination codon region” refer to a portion of such a mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the oligonucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of a mRNA (or corresponding nucleotides on the gene), and the 3′ untranslated region (3′UTR), the portion of a mRNA in the 3′ direction from the translation termination codon, including nucleotides between the translation termination codon and 3′ end of a mRNA (or corresponding nucleotides on the gene). The 5′ cap site of a mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap site.

For prokaryotic target nucleic acid, in various aspects, the nucleic acid is RNA transcribed from genomic DNA. For eukaryotic target nucleic acid, the nucleic acid is an animal nucleic acid, a plant nucleic acid, a fungal nucleic acid, including yeast nucleic acid. As above, the target nucleic acid is a RNA transcribed from a genomic DNA sequence. In certain aspects, the target nucleic acid is a mitochondrial nucleic acid. For viral target nucleic acid, the nucleic acid is viral genomic RNA, or RNA transcribed from viral genomic DNA.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an oligonucleotide-functionalized nanoparticle. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific oligonucleotide.

### EXAMPLES

### Example 1

### Preparation of Nanoparticles

Citrate stabilized gold nanoparticles (13±1 nm) were prepared using procedures known in the art. Thiolated oligonucleotide sequences, consisting of a block of nucleotide sequences, a poly adenine spacer, and a 3′-thiol modifier, were synthesized on an Expedite 8909 Nucleotide Synthesis System (ABI) using standard solid-phase synthesis and reagents (Glen Research) to create various oligonucleotide nanoparticles (oligo-NPs). Spacer Phosphoramidite C3 3-(4,4′-Dimethoxytrityloxy)propyl-1-[(2-cyanoethyl)-(N,N-diisopropyl)]-phosphoramidite (“C3” below) was used to provide a phosphate backbone lacking ribose and nucleobase components. dSpacer CE Phosphoramidite5′-O-Dimethoxytrityl-1′,2′-Dideoxyribose-3′-[(2-cyanoethyl)-(N,N-diisopropyl)]-phosphoramidite (“D” below) was used as a ribose which lacks the nucleobase component. (5′ CAG CTG CAC GCT GCC GTC T(A)10 SH-3′ (SEQ ID NO: 1)), (5′ (C3) CAG CTG CAC GCT GCC GTC (A)10 SH-3′ (SEQ ID NO: 2)), (5′ (C3)5 CAG CTG CAC GCT GC (A)10 St1-3′ (SEQ ID NO: 3)), (5′ CAG CTG CAC G(C3)5 CT GC (A)10 SH-3′ (SEQ ID NO: 4)), (5′CAG CTG(C3) CAC GCT GCC GTC (A)10 SH-3′ (SEQ ID NO: 5)), (5′ T5(C3)5 CAG CTG CAC (A) 10 SH-3′ (SEQ ID NO: 6)), (5′ (T)5 CAG CTG CAC GCT GC (A)10 SH-3′ (SEQ ID NO: 7)), (5′ (T)10 CAG CTG CAC (A)10 SH-3′ (SEQ ID NO: 8)), (5′ (T)19 (A)10 SH-3′ (SEQ ID NO: 9)), (5′ (T)30 SH-3′ (SEQ ID NO: 10)), (5′ (C)5 CAG CTG CAC GCT GC (A)10 SH-3′ (SEQ ID NO: 11)), (5′ (C)10 CAG CTG CAC (A)10 SH-3′ (SEQ ID NO: 12)), (5′ (C)19 (A)10 SH-3′ (SEQ ID NO: 13)), (5′ (C)30 SH-3′ (SEQ ID NO: 14)), (5′ (A)5 CAG CTG CAC GCT GC (A)10 SH-3′ (SEQ ID NO: 15)), (5′ (A)10 CAG CTG CAC (A)10 SH-3′ (SEQ ID NO: 16)), (5′ (A)19 (A)10 SH-3′ (SEQ ID NO: 17)), (5′ (A)30 SH-3′ (SEQ ID NO: 18)), (5′ (D)5 CAG CTG CAC GCT GC (A)10 SH-3′ (SEQ ID NO: 19)), (5′ (D)19 (A)10 SH-3′ (SEQ ID NO: 20)).

All sequences were HPLC purified following synthesis. The oligonucleotide-Au NPs were prepared using previously published methods. Briefly, thiol-modified oligonucleotides (3 μM) were added to Au NPs (10 nM) in Nanopure™ water (18.2 MΩ). The solution was brought to concentrations of 0.01% SDS, 0.01 M phosphate buffer pH 7.4, and 0.1M NaCl. The solution was further aged with additions of NaCl over 12 hours to bring the final NaCl concentration to 0.3M. Functionalized nanoparticles were separated from free oligonucleotides via three consecutive centrifugation steps (13,000 rpm, 20 min) and washed with phosphate buffered saline solution (PBS) (137 mM NaCl, 10 mM phosphate, 2.7 mM KCl, pH 7.4, Hyclone) after each centrifugation interval. Finally, the particles were re-suspended in PBS buffer and filter sterilized using a 0.2 μm acetate syringe filter (GE). Particle concentrations were determined by measuring extinction at 524 nm on a UV/visible spectrophotometer (Agilent Technologies). The particle DNA loading was determined fluorescently using a modification of literature procedures. Briefly, the set of oligonucleotide sequences (above) were synthesized with a fluorescein fluorophore on the 5′ terminus. Upon oxidative dissolution of the Au with KCN, the fluorescence was measured and correlated with a standard curve to determine DNA concentration.

### Example 2

### Cellular Uptake

The uptake of oligo-Au NPs was studied using a HcLa (human cervical carcinoma) cell line obtained from ATCC. Cells were maintained in Dulbecco's modified Eagle's medium (DMEM) supplemented with 10% heat-inactivated FBS and 1% penicillin/streptomycin at 5% CO₂ and 37° C. Sterile filtered oligo-NPs were added directly to the cell culture media of adherent cells at a concentration of 6 or 12 nM. Twenty four hours after nanoparticle addition, the cells were washed three times in phosphate buffered saline (PBS), collected with trypsin digestion, and counted using a Guava EasyCyte flow cytometer (Guava Technologies). To prepare samples for inductively coupled plasma mass spectrometry (ICP-MS) (Thermo-Fisher) to determine gold concentration, the cells were dissolved with neat nitric acid at 60° C. overnight. The number of 13 nm particles was determined by ICP-MS as previously described. All ICP experiments were preformed in triplicate and values obtained were averaged.

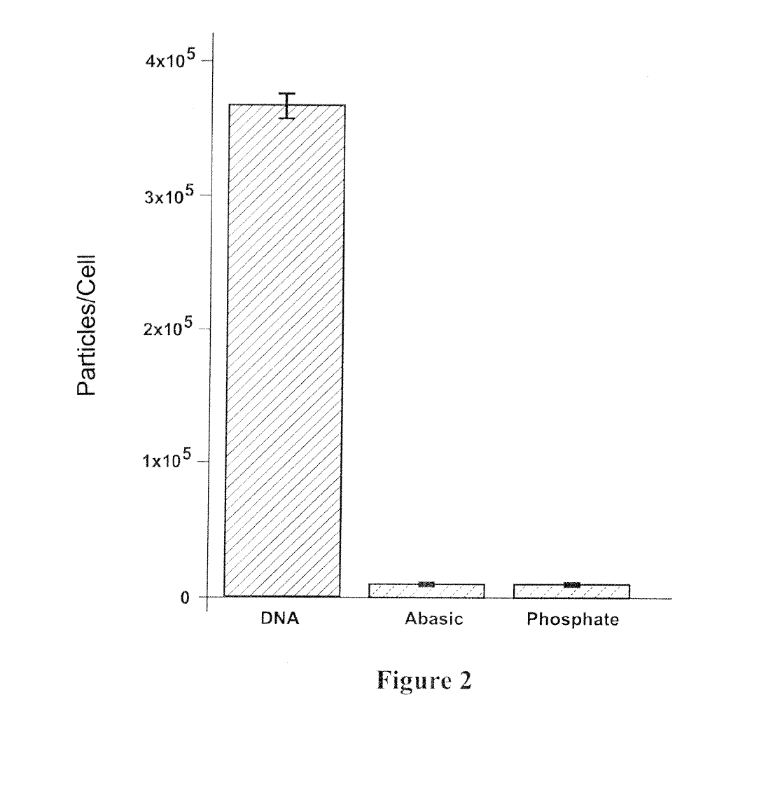

To determine the contributions of the oligonucleotide components to uptake, separate oligo-Au NP conjugates were made that included the phosphate backbone alone (C3), the phosphate backbone plus the ribose ring (abasic), and oligonucleotides complete with DNA nucleobases (DNA) (FIG. 1). Each sequence was designed to match with respect to net charge and oligonucleotide density. Oligonucleotide density on the Au NP was controlled by using a ten adenine spacer which allowed for consistent oligonucleotide loading. Measurements of oligonucleotide loading (strands/Au NP) and charge were obtained using fluorescence measurements and zeta potential, respectively (FIG. 1). A set of PEG particles was used as a negative control as these conjugates show comparatively little uptake in cells. Conjugates were added to cell culture and tested for cellular internalization. ICP measurements to monitor gold content confirm high uptake of particles with DNA functionalized nanoparticles, however the abasic and C3 sequences were not readily taken up by cells (similar to PEG controls, FIG. 2).

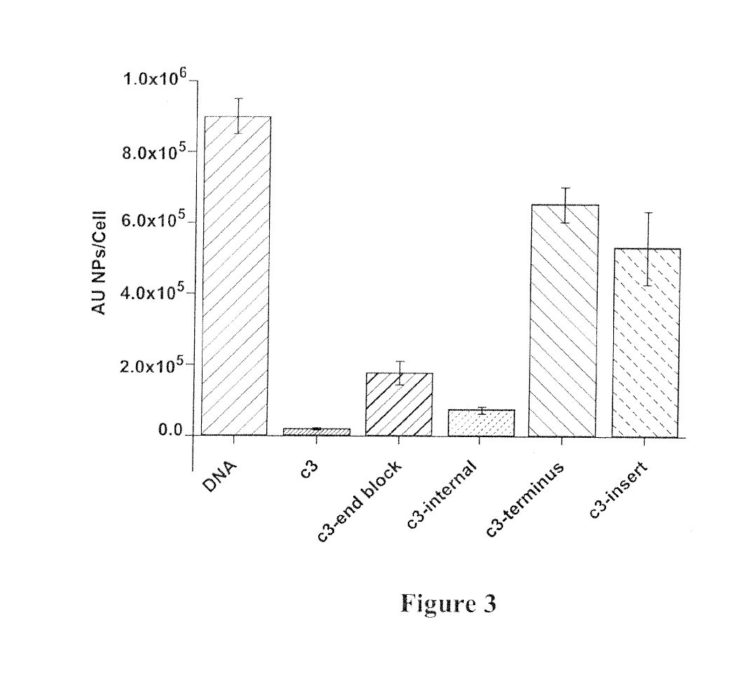

The effect of nucleobase positioning was then examined. Since one end of the oligonucleotide sequence is immobilized on the Au NP surface via gold-thiol bonding, the nanoparticle was used to spatially control interaction of the oligonucleotides with cells. Given that the C3 particles showed poor uptake into cells, the number and location of these residues was varied to determine the effects on cellular uptake. A single, or five block repeat, of C3 phosphoramadite was added either internally or as the terminal base of the oligonucleotide sequences. Addition of C3 residues to DNA strands decreases the uptake of the conjugates. The uptake scales with respect to the number of C3 residues (more C3 equals decreased uptake). The positioning of this C3 with respect to the DNA strand does not appear to play a significant role (FIG. 3). For example, placing a single C3 reside on the end of a DNA strand (C3 terminus) or at a position within the sequence (C3 insert), decreases its cellular uptake. Increasing the number of additional C3 residues to a five residue repeat (C3 end block, C3 internal) further decreases cellular uptake (FIG. 3).

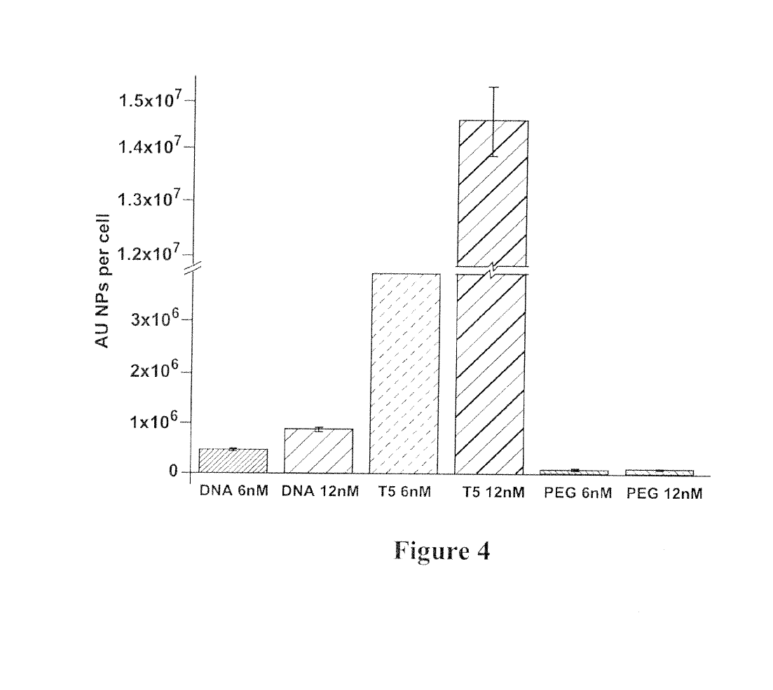

Given the necessity of DNA nucleobases for cellular uptake, over a dozen different DNA oligomers on the particle surface were examined. Different 19-base DNA combinations that contained deoxycytidine, deoxyguanosine, deoxyadenosine, thymidine residues did not show any significant changes in uptake as a result of DNA sequence. Repeats (n>5) of thymidine at the terminus of DNA strands, however, was found to increase cellular uptake of oligonucleotide-functionalized nanoconjugates by up to an order of magnitude (FIG. 4). Repeated residues of deoxycytidine and deoxyadenosine did not have this effect. Note that poly deoxyguanosine repeats were not synthesized due to the tendency of these structures to form G-quadraplexes and aggregate in solution.

### Example 3

### Protein Adsorption

Oligo-NPs (final concentration 6 nM) were incubated in serum-containing media for six hours at 37° C. After incubation, conjugates were isolated from solution via three consecutive centrifugation steps (13,000 rpm, 20 min) and washed with PBS to remove unbound proteins. Au NPs were dissolved with KCN (2.5 mM final concentration) and a Quant-iT fluorescence protein assay (Invitrogen) was used to determine the relative number of proteins in the solution. Estimation of the number of bound proteins per Au NP was calculated using a standard curve of bovine serum albumin (BSA) and an assumed average protein size of 60 kD.

DNA particles had been previously observed to adsorb proteins in media. Dynamic Light Scattering (DLS) data show that the average diameter of an Au NPs functionalized with DNA increase in size by over 30 nm upon exposure to cell culture media (Giljohann el al., Nano Lett. 7(12): 3818-3821, 2007, incorporated herein by reference in its entirety). This observation suggests that components of the cell culture media are attracted to the conjugates, and may be involved in mediating cellular uptake. Thus, whether the poor internalization of C3 and abasic conjugates was due to an inability to adsorb proteins was investigated. A similar number of proteins were found to be bound to these conjugates (approximately 20) when compared to their DNA counterparts.

These results show that the nucleobases are the contributing factor in the cellular uptake of these conjugates. Since the high charge density was matched in both the case of the C3 and abasic particles, the poor uptake of these structures eliminates charge as the critical component in the oligonucleotide internalization. All sets of conjugates showed similar numbers of serum proteins adsorbed on the particles. This observation suggests that protein adsorption is likely due to charge and is not a contributing factor to cellular recognition. The addition of specific DNA domains of poly thymine repeats was found to further increase the cellular uptake of polyvalent-oligonucleotide nanoparticle conjugates. The high internalization that results from poly thymine repeats is contemplated to further improve the cellular uptake of conjugates, and will serve as a method for increasing cellular internalization of oligonucleotides in general.

In summary, DNA-Au NPs have an extraordinary ability to enter cells. Their internalization is the result of several factors, including oligonucleotide density and the presence of nucleobases. Further, the positioning of DNA bases plays a role in cellular uptake, and the location of these residues allows one to modulate their interaction with cells. Specific repeated residues (poly thymidine) are used to affect cellular uptake.

While the present invention has been described in terms of various embodiments and examples, it is understood that variations and improvements will occur to those skilled in the art. Therefore, only such limitations as appear in the claims should be placed on the invention.

What is claimed is:

1. A nanoparticle functionalized with an oligonucleotide and a domain, the nanoparticle having the property of being taken up by a cell at an efficiency different than a nanoparticle functionalized with the same oligonucleotide but lacking the domain.

2. The nanoparticle of claim 1 wherein the domain is located 5′ to the oligonucleotide.

3. The nanoparticle of claim 1 wherein the domain is located 3′ to the oligonucleotide.

4. The nanoparticle of claim 1 wherein the domain is located at an internal region within the oligonucleotide.

5. The nanoparticle of any of the preceding claims wherein the domain is colinear with the oligonucleotide.

6. The nanoparticle of claim 1 functionalized with a second oligonucleotide and the domain is associated with the second oligonucleotide.

7. The nanoparticle of any of the preceding claims wherein the domain comprises a polythymidine (polyT) sequence comprising more than one thymidine residue.

8. The nanoparticle of any of the preceding claims wherein the domain comprises a polythymidine (polyT) sequence comprising two thymidine residues.

9. The nanoparticle of any of the preceding claims wherein the domain comprises a polythymidine (polyT) sequence comprising two, three, four, five, six, seven, eight, nine, ten, eleven, twelve, thirteen, fourteen, fifteen, sixteen, seventeen, eighteen, nineteen, or twenty thymidine residues.

10. The nanoparticle of any one of claims 1 through 6 wherein the domain comprises a phosphate polymer (C3 residue).

11. The nanoparticle of any one of claims 1 through 6 wherein the domain comprises two or more phosphate polymers (C3 residues).

12. A method of modulating cellular uptake capacity of an oligonucleotide-functionalized nanoparticle comprising the step of:
modifying the nanoparticle to further comprise a domain that modulates cellular uptake of the oligonucleotide-functionalized nanoparticle compared to the oligonucleotide-functionalized nanoparticle lacking the domain.

13. The method of claim 12 wherein the domain increases cellular uptake of the functionalized nanoparticle.

14. The method of claims 12 or 13 wherein the domain comprises a polyT sequence comprising more than one thymidine residue.

15. The method of any one of claims 12 through 14 wherein the domain comprises a polyT sequence comprising two, three, four, five, six, seven, eight, nine, ten, eleven, twelve, thirteen, fourteen, fifteen, sixteen, seventeen, eighteen, nineteen, or twenty thymidine residues.

16. The method of any one of claims 12 through 15 wherein an increase in thymidine residues in the polyT sequence of the first oligonucleotide-functionalized nanoparticle increases cellular uptake compared to the second oligonucleotide-functionalized nanoparticle that does not contain the polyT sequence.

17. The method of any one of claims 12 through 16 wherein the domain decreases cellular uptake of the oligonucleotide-functionalized nanoparticle.

18. The method of claim 12 wherein the domain comprises a phosphate polymer (C3 residue).

19. The method of claim 18 wherein an increase in C3 residues on the first oligonucleotide-functionalized nanoparticle decreases cellular uptake compared to the second oligonucleotide-functionalized nanoparticle that does not contain a C3 residue.

20. The method of any one of claims 12 through 19 wherein the oligonucleotide-functionalized nanoparticle is the nanoparticle of any one of claims 1 through 11.
